(19) United States
(12) Patent Application Publication
(10) Pub. No.: US 2023/0144187 A1
(43) Pub. Date: May 11, 2023

(54) PREPARATION METHOD OF THERMOSENSITIVE GEL FOR TREATING ANDROGENETIC ALOPECIA

(71) Applicant: The First Hospital of Lanzhou University, Lanzhou City (CN)

(72) Inventors: Jia YAO, Lanzhou City (CN); Yongcui MAO, Lanzhou City (CN); Pinyan LIU, Lanzhou City (CN)

(21) Appl. No.: 17/808,122

(22) Filed: Jun. 22, 2022

(30) Foreign Application Priority Data
Nov. 5, 2021 (CN) 202111308771.3

(51) Int. Cl. A61K 35/28; A61K 9/19; A61K 9/06; A61P 17/14

(52) U.S. Cl. CPC A61K 35/28; A61K 9/19; A61K 9/06; A61P 17/14

(57) ABSTRACT

The present disclosure provides a preparation method of a thermosensitive gel for treating androgenetic alopecia. The preparation method includes the following steps: step 1, separating umbilical cord mesenchymal stem cells (UC-MSCs); step 2, conducting adherent culture of the UC-MSCs; step 3, preparing a lyophilized powder of umbilical cord-mesenchymal stem cell-conditioned medium (UC-MSC-CM) supernatant; and step 4, preparing the thermosensitive gel. In the present disclosure, the thermosensitive gel is prepared by using the UC-MSC-CM supernatant. Based on an important role of cytokines encapsulated in exosomes secreted by the UC-MSCs in the hair growth process, the exosomes are encapsulated in the thermosensitive gel for topical use in treating androgenetic alopecia, featuring easy operation, convenient treatment, and excellent efficacy.

### CROSS REFERENCE TO RELATED APPLICATION(S)

This patent application claims the benefit and priority of Chinese Patent Application No. 202111308771.3, filed on Nov. 5, 2021, the disclosure of which is incorporated by reference herein in its entirety as part of the present application.

### TECHNICAL FIELD

The present disclosure belongs to the technical field of alopecia, and in particular relates to a preparation method of a thermosensitive gel for treating androgenetic alopecia.

### BACKGROUND ART

With the rapid development of society, people's life pressure and work stress are increasing, and the prevalence of androgenetic alopecia (AGA) is increasing year by year. Although AGA does not affect physical health, it seriously affects the mental health and quality of life of patients. Although drug therapy for AGA has certain efficacy, AGA may be aggravated after drug withdrawal, and long-term use will have serious side effects. At present, some studies have used the supernatant of stem cell conditioned medium to be injected or topically applied to the alopecic area to promote hair growth. Researchers further found that the exosomes secreted by stem cells into the culture medium played a role.

However, due to the limited amount of exosomes secreted by stem cells in conditioned medium, the active ingredients in exosomes lose their activity too quickly, and frequent administration is required, which brings difficulties to clinical applications.

### SUMMARY

In view of the above problems, the present application sets forth a preparation method of a thermosensitive gel for treating androgenetic alopecia.

A technical solution used in the present disclosure is as follows:

a preparation method of a thermosensitive gel for treating androgenetic alopecia, including the following steps:

step 1: separating umbilical cord mesenchymal stem cells (UC-MSCs);

step 2: conducting adherent culture of the UC-MSCs;

step 3: preparing a lyophilized powder of umbilical cord-mesenchymal stem cell-conditioned medium (UC-MSC-CM) supernatant; and

step 4: preparing the thermosensitive gel.

Preferably, in step 1, the separating UC-MSCs may include the following steps:

step 101: collection of an umbilical cord: placing the umbilical cord in an umbilical cord storage bottle through aseptic operation, and immediately transporting the umbilical cord to a laboratory for treatment at a constant temperature of 4° C.;

step 102: alcohol disinfection: placing the umbilical cord in a beaker and pouring 75% alcohol to immerse the umbilical cord for disinfecting for 10-15 s, transferring the umbilical cord to another beaker, adding 50 mL of normal saline for rinsing, discarding washings after rinsing, and repeating 2 to 3 times;

step 103: removal of blood stasis: using surgical scissors and hemostatic forceps to cut the umbilical cord into 5-10 segments with a length of 0.5-1 cm in a Petri dish containing 50 mL of normal saline, removing blood clots, putting the excess back into the umbilical cord storage bottle for later use, putting the cut umbilical cord into a sterile beaker with the hemostatic forceps, cleaning the cut umbilical cord with normal saline 2-3 times, removing blood stasis residues and mucus adhering to the surface, and discarding washings;

step 104: mincing: placing a cleaned umbilical cord in a sterile beaker, and using sterile surgical scissors to mince the umbilical cord into small pieces, where each piece is 2 mm³ in size;

step 105: attaching: inoculating minced umbilical cord tissue pieces evenly into at least three T75 culture flasks with a sterile long spoon, where at least 200 pieces are attached in each flask;

step 106: drying: putting the T75 culture flasks attached with the umbilical cord tissue pieces into a humidified 5% CO₂ incubator at 37° C., and letting the T75 culture flasks stand for 3 h;

step 107: addition of a culture medium: after letting stand for 3 h, adding 15 mL of a well-prepared culture medium to each attached tissue culture flask in a clean bench, and putting a T75 culture flask added with the culture medium into the humidified 5% CO₂ incubator at 37° C. for further culture;

step 108: medium change for culture: culturing for 6 days, observing and recording cell growth by Day 7, discarding an original culture medium, pipetting a new culture medium into the T75 culture flasks, and adding 15 mL of the culture medium to each flask; and

step 109: specimen passage: culturing for 9 days, observing the cell growth by Day 10, handing over in advance on condition that cells grow too dense after the medium change; conducting a second treatment of the umbilical cord on condition that there is no cell growth after the medium change.

Preferably, in step 2, the adherent culture of the UC-MSCs may include the following steps:

step 201: cleaning and disinfecting a clean bench, preparing reagents and materials required for an experiment, wiping an outer surface of a culture flask with 75% alcohol from a cap downward, and putting the culture flask into the clean bench immediately;

step 202: microscopically observing cell morphology as spindle-shaped and compact with circinate growth, and observing whether cell confluence reaches 70%-80%, and conducting subculture and multiplication culture on condition that requirements are meet;

step 203: aspirating the culture medium from the culture flask, adding 15-20 mL of sterile normal saline or Dhanks thereto for washing, and aspirating the normal saline or Dhanks to discard into a waste fluid tank; adding pancreatin for digestion; microscopically observing the digestion of cells, terminating the digestion after the cells are curled into a circle, preparing digested cells into a single cell suspension, and adding the single cell suspension to a 50 mL centrifuge tube; before centrifugation, counting cells from 200 μL of the cell suspension, and centrifuging the single cell suspension at 1,000 r/min for 5 min; and

step 204: aspirating and discarding a supernatant, and inoculating the cells at a density of 1-2×10⁶/T75 or 2-6×10⁶/T175 according to a cell count result; after inoculating, labeling and checking information regarding barcode, cell passage number, number of inoculated flasks, and operation date on a T75 or T175 cell culture flask, and putting the flask into a CO₂ incubator at 37° C. and 5% CO₂ for culture.

Preferably, in step 202, the subculture may be conducted when the cell confluence reaches 80%.

Preferably, in step 3, the UC-MSCs in step 2 may be cultured in serum-free basal medium for 24-48 h, a cell supernatant may be collected, cell debris may be discarded after centrifugation, cells may be counted and dispensed in a beaker, placed at a temperature of −50° C. to 80° C. for freezing treatment, and lyophilized at −50° C. to obtain a lyophilized powder of mesenchymal stem cell supernatant.

Preferably, in step 4, the lyophilized powder of mesenchymal stem cell supernatant may be dissolved in 4° C. triple distilled water, added to a gel matrix for standing, and left stand in a refrigerator at 4° C. for 24 h to form a gel system.

Preferably, each matrix in the gel system may have the following volume ratio: 20% poloxamer 407, 4% poloxamer 188, 8% lyophilized powder of mesenchymal stem cell supernatant, and the balance being 4° C. triple distilled water.

The present disclosure has the following beneficial effects:

1. Based on an important role of cytokines encapsulated in exosomes secreted by the UC-MSCs in the hair growth process, the exosomes are encapsulated in the thermosensitive gel for topical use in treating androgenetic alopecia, featuring easy operation, convenient treatment, and excellent efficacy.

2. As a carrier of protein components, thermosensitive hydrogel may significantly slow down the degradation rate of protein components, and improve the bioavailability of active components in conditioned medium by means of sustained-release effect.

3. The method may respond in time to the change of outside ambient temperature and realize the phase transition of solution-gel. With the degradation or dissolution of the gel on the body surface, the drug is slowly released, which may avoid the first-pass effect of the active ingredient and prolong residence time of active ingredients in therapeutic areas, thus achieving the objective of continuous administration.

### BRIEF DESCRIPTION OF THE DRAWINGS

In order to explain the examples of the present disclosure or the technical solutions in the prior art more clearly, the accompanying drawings that need to be used in the description of the examples or the prior art will be briefly introduced below. Obviously, the accompanying drawings in the following description are only some examples of the present disclosure. Other drawings can also be obtained by those of ordinary skill in the art based on these drawings without creative efforts.

FIG. 1 illustrates a lyophilized powder of UC-MSC supernatant;

FIG. 2 illustrates a stem cell hair growth gel;

FIG. 3 shows data analysis charts of animal hair diameter and hair length obtained from animal experiments.

### DETAILED DESCRIPTION OF THE EMBODIMENTS

In order to make the objectives, technical solutions and advantages of the examples of the present disclosure clearer, the technical solution in the examples of the present disclosure will be clearly and completely described below with reference to the accompanying drawings in the examples of the present disclosure. Obviously, the described embodiments are a part of, not all of, examples of the present disclosure. The components of the examples of the present disclosure generally described and illustrated in the drawings herein may be arranged and designed in a variety of different configurations.

Thus, the following detailed description of the examples of the present disclosure provided in the accompanying drawings is not intended to limit the scope of the present disclosure as claimed, but is merely representative of selected examples of the present disclosure. Based on the examples of the present disclosure, all other examples obtained by those of ordinary skill in the art without creative efforts shall fall within the protection scope of the present disclosure.

This example specifically provided a preparation method of a thermosensitive gel for treating androgenetic alopecia, including the following steps:

step 1: separation of UC-MSCs;

step 2: adherent culture of the UC-MSCs;

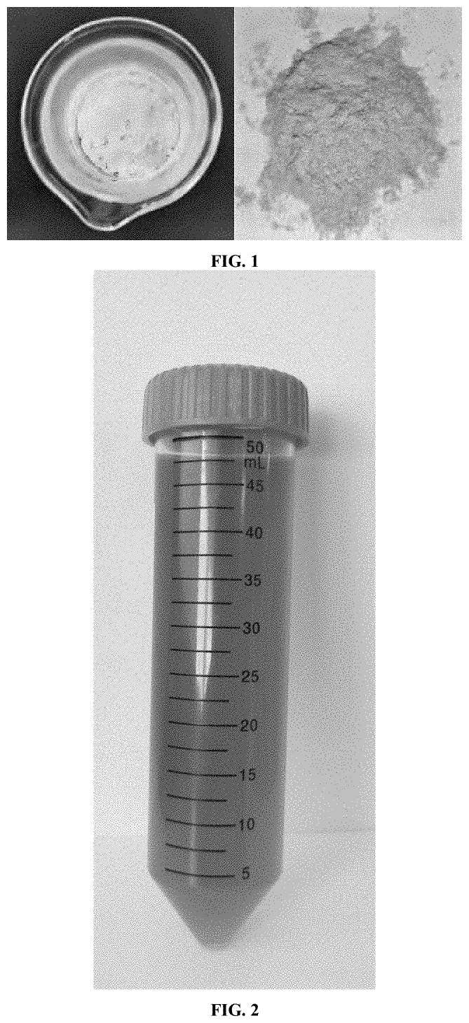

step 3: preparation of a lyophilized powder of UC-MSC-CM supernatant: the UC-MSCs in step 2 were cultured in serum-free basal medium for 24-48 h, a cell supernatant was collected, cell debris was discarded after centrifugation, cells were counted and dispensed in a beaker, placed at a temperature of −50° C. to 80° C. for freezing treatment, and lyophilized at −50° C. to obtain a lyophilized powder of mesenchymal stem cell supernatant shown in FIG. 1;

step 4: preparation of the thermosensitive gel: the lyophilized powder of mesenchymal stem cell supernatant was dissolved in 4° C. triple distilled water, added to a gel matrix, and left stand in a refrigerator at 4° C. for 24 h to form a gel system; each matrix in the gel system had the following volume ratio: 20% poloxamer 407, 4% poloxamer 188, 8% lyophilized powder of mesenchymal stem cell supernatant, and the balance being 4° C. triple distilled water. The result is shown in FIG. 2.

In step 1, the separation of UC-MSCs included the following steps:

step 101: collection of an umbilical cord: the umbilical cord was placed in an umbilical cord storage bottle through aseptic operation, and immediately transported to a laboratory for treatment at a constant temperature of 4° C.;

step 102: alcohol disinfection: the umbilical cord was placed in a beaker and 75% alcohol was poured to immerse the umbilical cord for disinfecting for 10 s; the umbilical cord was transferred to another beaker, 50 mL of normal saline was added for rinsing, washings were discarded after rinsing, and the step was repeated 2 to 3 times;

step 103: removal of blood stasis: surgical scissors and hemostatic forceps were used to cut the umbilical cord into 5-10 segments with a length of 0.5-1 cm in a Petri dish containing 50 mL of normal saline, blood clots were removed, and the excess was put back into the umbilical cord storage bottle for later use; the cut umbilical cord was put into a sterile beaker with the hemostatic forceps, and cleaned with normal saline 2-3 times; blood stasis residues and mucus adhering to the surface were removed, and washings were discarded;

step 104: mincing: a cleaned umbilical cord was placed in a sterile beaker, and the umbilical cord was minced into small pieces using sterile surgical scissors, where each piece was 2 mm³ in size;

step 105: attaching: minced umbilical cord tissue pieces were inoculated evenly into at least three T75 culture flasks with a sterile long spoon, where at least 200 pieces were attached in each flask;

step 106: drying: the T75 culture flasks attached with the umbilical cord tissue pieces were put into a humidified 5% CO₂ incubator at 37° C., and let stand for 3 h;

step 107: addition of a culture medium: after letting stand for 3 h, 15 mL of a well-prepared culture medium (components of the culture medium: DMEM/F12:FB S=9:1) was added to each attached tissue culture flask in a clean bench, and a T75 culture flask added with the culture medium was put into the humidified 5% CO₂ incubator at 37° C. for further culture;

step 108: medium change for culture: after culturing for 6 days, cell growth was observed and recorded by Day 7, an original culture medium was discarded, a new culture medium was pipetted into the T75 culture flasks, and 15 mL of the culture medium was added to each flask; and

step 109: specimen passage: after culturing for 9 days, the cell growth was observed by Day 10; handing over was conducted in advance on condition that cells grew too dense after the medium change; a second treatment of the umbilical cord was conducted on condition that there was no cell growth after the medium change.

In step 2, the adherent culture of the UC-MSCs included the following steps:

step 201: a clean bench was cleaned and disinfected, reagents and materials required for an experiment were prepared, an outer surface of a culture flask was wiped with 75% alcohol from a cap downward, and the culture flask was put into the clean bench immediately;

step 202: cell morphology was microscopically observed as spindle-shaped and compact with circinate growth, and whether cell confluence reached 70%-80% was observed, and subculture and multiplication culture were conducted on condition that requirements were meet;

step 203: the culture medium was aspirated from the culture flask, 15-20 mL of sterile normal saline or Dhanks was added thereto for washing, and the normal saline or Dhanks was aspirated to discard into a waste fluid tank; pancreatin was added for digestion; the digestion of cells was microscopically observed, the digestion was terminated after the cells were curled into a circle; digested cells were prepared into a single cell suspension, and added to a 50 mL centrifuge tube; before centrifugation, cells were counted from 200 μL of the cell suspension, and the single cell suspension was centrifuged at 1,000 r/min for 5 min; and

step 204: a supernatant was aspirated and discarded, and the cells were inoculated at a density of 1-2×10⁶/T75 or 2-6×10⁶/T175 according to a cell count result; after inoculating, information regarding barcode, cell passage number, number of inoculated flasks, and operation date was labeled and checked on a T75 or T175 cell culture flask, and the flask was put into a CO₂ incubator at 37° C. and 5% CO₂ for culture.

In step 202, the subculture was conducted when the cell confluence reached 80%.

Animal Experiment

OBJECTIVE: To verify whether a stem cell hair growth gel has a promoting effect on androgenetic alopecia model mice.

METHODS: Fifteen C57BL6 mice, aged 6 weeks, were randomly divided into 3 groups after one-week adaptive feeding.

Blank group: After anesthesia, depilation was performed with rosin and paraffin (1:1), with a depilation area of about 2×3 cm, and no other treatment was performed.

Model group: Depilation was performed with rosin and paraffin, with a depilation area of 2×cm. On day 2 of depilation, dihydrotestosterone was intraperitoneally injected at a dose of 1 mg/time, 5 times a week, to establish an androgenetic alopecia model.

Experimental group: On the basis of the model group, the hair growth gel was applied twice a day, 1 mL each time, and the administration period was 17 days.

After the experiment, the mice were sacrificed by cervical dislocation; 100 hairs were randomly selected from each mouse to measure the diameter and length of the hair, and the data were analyzed for significant differences with Graph Pad Prism software. The difference was statistically significant at the detection level P<0.05.

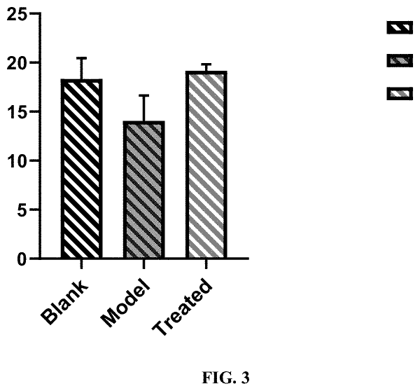

CONCLUSION: As shown in FIG. 3, compared with the blank group, the hair diameter of mice was significantly reduced (*P<0.05); compared with the experimental group, the hair diameter was significantly increased in the model group (*P<0.05); compared with the experimental group, the hair length was significantly increased in the model group (*P<0.05).

The above is only intended to illustrate the technical solution of the present disclosure, but not to limit it. Other modifications or equivalent substitutions made by those of ordinary skill in the art to the technical solution of the present disclosure should be included within the scope of the claims of the present disclosure as long as they do not depart from the spirit and scope of the technical solution of the present disclosure.

What is claimed is:

1. A preparation method of a thermosensitive gel for treating androgenetic alopecia, comprising the following steps:
step 1: separating umbilical cord mesenchymal stem cells (UC-MSCs);
step 2: conducting adherent culture of the UC-MSCs;
step 3: preparing a lyophilized powder of umbilical cord-mesenchymal stem cell-conditioned medium (UC-MSC-CM) supernatant; and
step 4: preparing the thermosensitive gel.

2. The preparation method of a thermosensitive gel for treating androgenetic alopecia according to claim 1, wherein in step 1, the separating UC-MSCs comprises the following steps:
step 101: collection of an umbilical cord: placing the umbilical cord in an umbilical cord storage bottle through aseptic operation, and immediately transporting the umbilical cord to a laboratory for treatment at a constant temperature of 4° C.;
step 102: alcohol disinfection: placing the umbilical cord in a beaker and pouring 75% alcohol to immerse the umbilical cord for disinfecting for 10-15 s, transferring the umbilical cord to another beaker, adding 50 mL of normal saline for rinsing, discarding washings after rinsing, and repeating 2 to 3 times;
step 103: removal of blood stasis: using surgical scissors and hemostatic forceps to cut the umbilical cord into 5-10 segments with a length of 0.5-1 cm in a Petri dish containing 50 mL of normal saline, removing blood clots, putting the excess back into the umbilical cord storage bottle for later use, putting the cut umbilical cord into a sterile beaker with the hemostatic forceps, cleaning the cut umbilical cord with normal saline 2-3 times, removing blood stasis residues and mucus adhering to the surface, and discarding washings;
step 104: mincing: placing a cleaned umbilical cord in a sterile beaker, and using sterile surgical scissors to mince the umbilical cord into small pieces, wherein each piece is 2 mm³ in size;
step 105: attaching: inoculating minced umbilical cord tissue pieces evenly into at least three T75 culture flasks with a sterile long spoon, wherein at least 200 pieces are attached in each flask;
step 106: drying: putting the T75 culture flasks attached with the umbilical cord tissue pieces into a humidified 5% CO₂ incubator at 37° C., and letting the T75 culture flasks stand for 3 h;
step 107: addition of a culture medium: after letting stand for 3 h, adding 15 mL of a well-prepared culture medium to each attached tissue culture flask in a clean bench, and putting a T75 culture flask added with the culture medium into the humidified 5% CO₂ incubator at 37° C. for further culture;
step 108: medium change for culture: culturing for 6 days, observing and recording cell growth by Day 7, discarding an original culture medium, pipetting a new culture medium into the T75 culture flasks, and adding 15 mL of the culture medium to each flask; and
step 109: specimen passage: culturing for 9 days, observing the cell growth by Day 10, handing over in advance on condition that cells grow too dense after the medium change; conducting a second treatment of the umbilical cord on condition that there is no cell growth after the medium change.

3. The preparation method of a thermosensitive gel for treating androgenetic alopecia according to claim 1, wherein in step 2, the adherent culture of the UC-MSCs comprises the following steps:
step 201: cleaning and disinfecting a clean bench, preparing reagents and materials required for an experiment, wiping an outer surface of a culture flask with 75% alcohol from a cap downward, and putting the culture flask into the clean bench immediately;
step 202: microscopically observing cell morphology as spindle-shaped and compact with circinate growth, and observing whether cell confluence reaches 70%-80%, and conducting subculture and multiplication culture on condition that requirements are meet;
step 203: aspirating the culture medium from the culture flask, adding 15-20 mL of sterile normal saline or Dhanks thereto for washing, and aspirating the normal saline or Dhanks to discard into a waste fluid tank; adding pancreatin for digestion; microscopically observing the digestion of cells, terminating the digestion after the cells are curled into a circle, preparing digested cells into a single cell suspension, and adding the single cell suspension to a 50 mL centrifuge tube; before centrifugation, counting cells from 200 μL of the cell suspension, and centrifuging the single cell suspension at 1,000 r/min for 5 min; and
step 204: aspirating and discarding a supernatant, and inoculating the cells at a density of 1-2×10⁶/T75 or 2-6×10⁶/T175 according to a cell count result; after inoculating, labeling and checking information regarding barcode, cell passage number, number of inoculated flasks, and operation date on a T75 or T175 cell culture flask, and putting the flask into a CO₂ incubator at 37° C. and 5% CO₂ for culture.

4. The preparation method of a thermosensitive gel for treating androgenetic alopecia according to claim 3, wherein in step 202, the subculture is conducted when the cell confluence reaches 80%.

5. The preparation method of a thermosensitive gel for treating androgenetic alopecia according to claim 1, wherein in step 3, the UC-MSCs in step 2 are cultured in serum-free basal medium for 24-48 h, a cell supernatant is collected, cell debris is discarded after centrifugation, cells are counted and dispensed in a beaker, placed at a temperature of −50° C. to 80° C. for freezing treatment, and lyophilized at −50° C. to obtain a lyophilized powder of mesenchymal stem cell supernatant.

6. The preparation method of a thermosensitive gel for treating androgenetic alopecia according to claim 1, wherein in step 4, the lyophilized powder of mesenchymal stem cell supernatant is dissolved in 4° C. triple distilled water, added to a gel matrix for standing, and left stand in a refrigerator at 4° C. for 24 h to form a gel system.

7. The preparation method of a thermosensitive gel for treating androgenetic alopecia according to claim 6, wherein each matrix in the gel system has the following volume ratio: 20% poloxamer 407, 4% poloxamer 188, 8% lyophilized powder of mesenchymal stem cell supernatant, and the balance being 4° C. triple distilled water.
